(19) United States
(12) Patent Application Publication
(10) Pub. No.: US 2009/0124982 A1
(43) Pub. Date: May 14, 2009

(54) Injector Device for Syringes

(75) Inventor: Luis Prieto Jimenez, Valencia (ES)

Correspondence Address: HONIGMAN MILLER SCHWARTZ & COHN LLP, 38500 WOODWARD AVENUE, SUITE 100, BLOOMFIELD HILLS, MI, 48304-5048, US

(73) Assignee: Innovaciones en Suministro de Nurticion Enteral, S.L., Valencia (ES)

(21) Appl. No.: 11/994,623

(22) PCT Filed: Jun. 15, 2006
(86) PCT No.: PCT/ES2006/000352

§ 371 Date: Jun. 23, 2008

(30) Foreign Application Priority Data
Jul. 5, 2005 (ES) U200501550

(51) Int. Cl. A61M 5/315

(52) U.S. Cl. 604/228

(57) ABSTRACT

The invention relates to an injector device for a syringe, the syringe including a plunger movably receivable by a barrel, the plunger having a push flange and the barrel defining an interior to movably receive the plunger, the barrel further including a base end including one or more lateral flanges and a tip end, the injector device comprising a first support defining a cavity surrounded by a peripheral edge into which the barrel of the syringe is perpendicularly insertable, wherein the base end of the barrel is supportable by the engagement between the peripheral edge of the first support and the one or more lateral flanges of the base end of the barrel, a second support movably coupled to the first support, the second support having a guide surface that abuttably engages the push flange of the plunger when engaged therewith, and at least two parallel guides movably coupling the second support to the first support.

### TECHNICAL FIELD

The present invention relates to an application for syringes, particularly for those syringes used to introduce fluids of a certain density into the body.

The object of the invention is to shape the device with a simple structure, manually operable, and that is provided with means to facilitate the gradual movement of the syringe plunger with barely any effort required on the part of the patient or a third person.

### BACKGROUND

Due to an ageing population and developments in the field of medicine, the situation has arisen in which the need for sanitary assistance at home is increasingly greater.

One of the complications or consequences of a wide group of illnesses consists in the alteration of deglutition or dysphagia, which determines the need to place, for example, nasogastric tubes that guarantee that the patients are fed. Illnesses that can cause dysphagia include, amongst others, degenerative illnesses such as dementia, patients having undergone a tracheotomy, neuromuscular and oncological illnesses.

In the tube feeding process, the ground up food is generally administered through the tube with a density higher than that of water, syringes of considerable dimensions thus being used for this purpose. For this reason, the pressure required to move the syringe plunger in order to be able to inject the food through the nasogastric tube is very high. This can consequently generate great difficulty for the patient due to the force that must be exerted on the transversal flanges of the syringe that is already of a reduced size. Furthermore, if we add the fact that this concerns procedures that are carried out on a daily basis and several times per day, repeated microtraumas may be caused that will have clinical repercussions over time and moreover that will have a far greater probability of occurring in the case of individuals with rheumatic processes. Furthermore, the pain it can cause to a patient's fingers and hand must be taken into consideration.

Likewise, there are cases of advanced ailments that require the participation of an assistant to provide the patient with the diet required.

Certain patents related to the technical domain of this invention are described in the following.

The Invention Patent ES 2 176 457 describes a syringe for the dosed injection of a medicine which intends to facilitate the operation of said syringe through the use of an interchangeable cartridge, of the type that contains a piston forced into a tubular cartridge in order to eject a dose corresponding to the movement of the piston.

The Invention Patent ES 2 128 694 describes an injector, operated mechanically and controlled manually, that incorporates a pistol-type handle and houses in its interior a motor associated to a screw that determines the movement of the syringe plunger.

The Invention Patent ES 1 055 061 refers to a syringe that includes a cylinder that houses a plunger that is axially displaceable in said cylinder, related by means of one or more elastic traction elements that constantly drive said plunger towards the cylinder base. The distance between the anchorage points of the piston where the elastic elements are anchored is variable, offering the possibility of selecting different anchorage points that produce a different extension of the elastic elements and therefore different pushing pressures.

### SUMMARY

The present invention relates to an injector device for syringes that aims to reduce the force exerted by the fingers when moving the plunger of a syringe by incorporating a mechanism for such a purpose that offers a greater support surface so that in this way it is possible to reduce the application pressure necessary, without straining the hand or the joints, and to allow the patient him/herself to inject him/herself in a way simple without the need for external help.

The injector device consists of a first support and a second support that moves in a guided way on the parallel guides with respect to the first support.

The first support presents a cavity that is crossed by the cylindrical barrel of the syringe, said cavity being delimited by a peripheral edge on which the flanges that make up the base of the barrel of the syringe rest. This cavity can consist, in a possible embodiment, in a U-shaped section made on one side of the first support or, for example, it can be a circular opening through which the barrel of the syringe is introduced by its tip and that presents a slightly larger diameter than that of the barrel.

The base of the plunger or push flange rests on one of the faces of the second moveable support, in this way being arranged so that when pressure is applied with the hand on said second support, while holding the fixed position of the first support, the plunger is forced to advance and will gradually penetrate the cylinder pushing the viscous fluid contained in its interior in the direction towards the tube, for example. The wide surface with which the second support is provided allows both hands to be applied on said second support with the thumbs supported on the external face of the first support, directing the plunger with barely any resistance towards the interior of the cylinder, thus proving particularly useful for fluids of a certain density.

The maximum run of the second support is defined by the distance existing between the first support and the final end of the parallel guides that start at said first support. Optionally, provision has been made for the second support to incorporate, into its wall opposite the first support, a recess into which the base of the plunger or push flange is inserted, in this way facilitating positional stability during the run of the plunger and avoiding undesired movement.

Respective stoppers are found on the final ends of the parallel guides that will prevent the escape of the second support and will determine its maximum run as mentioned previously, said stoppers could be substituted with a third support.

Therefore, in accordance with this structuring of the device proposed, it is possible for the patient to introduce dense food by means of this syringe towards a tube while only exerting minimum pressure.

### DESCRIPTION OF THE DRAWINGS

In order to complement the description being given and with the object of aiding the better comprehension of the characteristics of the invention, in accordance with a preferred example of a practical embodiment of the latter, a set of drawings is included as an integral part of said description which is of an illustrative and not limitative character, in which the following is disclosed:

FIG. 1.—Shows a perspective view in which it is possible to see the device that is the object of the invention without the syringe.

FIG. 2.—Shows a perspective view of the injector device in which the syringe is seen mounted for the maximum extension position of the plunger.

FIG. 3.—Shows a perspective view of the injector device for an intermediate position during the compression of the syringe plunger by means of the action of the injector device.

FIG. 4.—Shows a perspective view of a feature of the injector device with the syringe mounted, in which is it possible to see the flanges of the syringe resting on the peripheral edge of the section of the first support.

### PREFERRED EMBODIMENT

With reference to the Figures a description is given hereafter of a preferred embodiment of the injector device for syringes that constitutes the object of the invention, in an application for syringes that conventionally consist of an essentially cylindrical barrel (1), provided with at least one flange (2) on its base, on the interior of which a plunger (3) is moved that has a push flange (4) at its base.

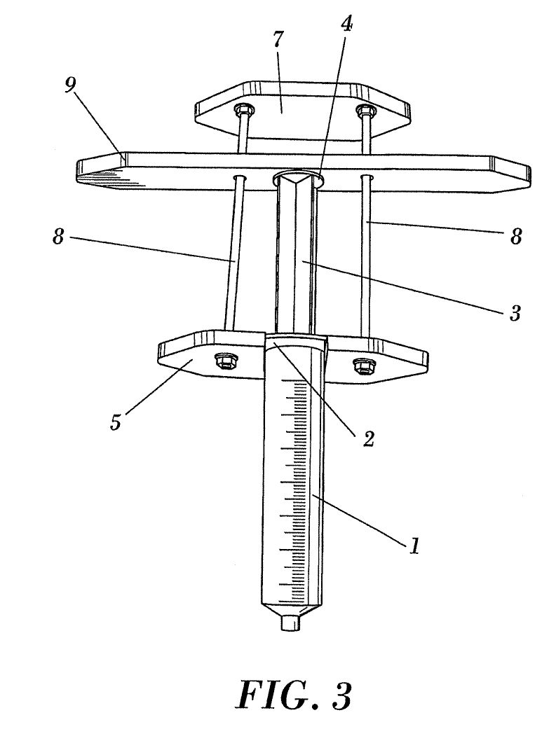

As can be seen in FIG. 3 the injector device is made up of:
a first support (5) provided with a cavity (6) in which the barrel of the syringe (1) is inserted perpendicularly to said first support (5), said syringe resting by means of the flanges (2) on the peripheral edge of said cavity (6) on a internal wall of the first support (5),
a second support (9) against which the push flange (4) of the syringe rests and that moves guided by manual action on some parallel guides (8), that start from the first support (5), between said first support (5) and the final ends of said parallel guides (8) thus pushing the plunger (3) in the interior of the cylindrical barrel (1).

The final ends of the parallel guides (8) can be finished with respective stoppers that prevent the accidental escape of the second support (9) by delimiting its maximum run or alternatively a third support (7) parallel to the first support (5) can be coupled to said final ends.

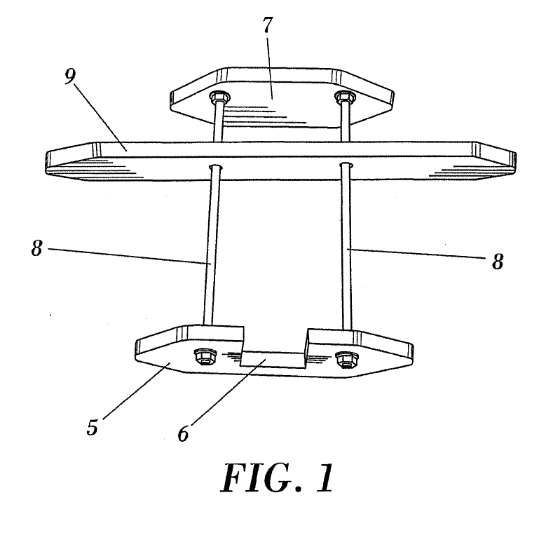

The cavity (6) can consist, as represented in FIG. 1, of a U-shaped section made on one of the sides of the first support (5).

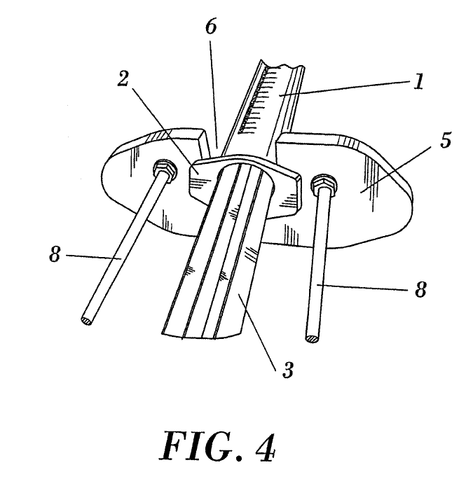

In FIG. 4 it is possible to observe the way in which the barrel (1) of the syringe supported by its flanges (2) on the first support (5) is positioned.

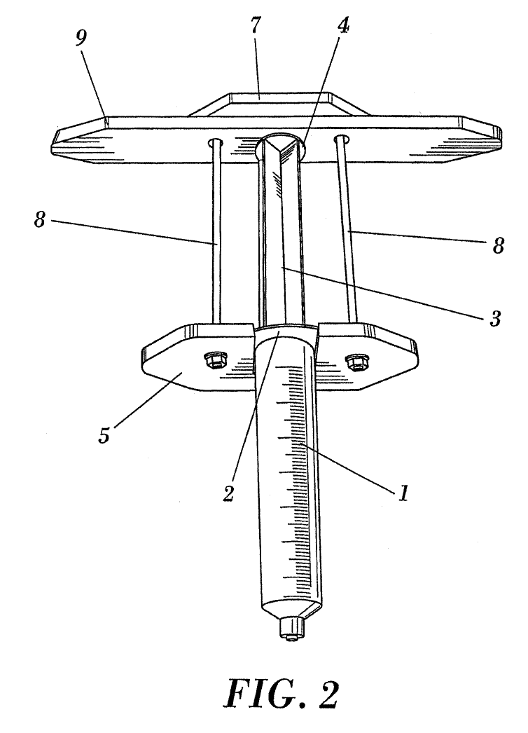

In FIGS. 2 and 3 it is possible to see two working sequences of the device that is the object of this invention, in FIG. 2 the plunger (3) appears completely extended, and then, as pressure is exerted on the second support (9) by means of the use of the hands, holding the first support in a fixed position (5), said second support (9) is moved with very little effort to push the plunger (3) towards the interior of the barrel (1), as can be seen in FIG. 3.

1-6. (canceled)

7. An injector device for a syringe, the syringe including a plunger movably receivable by a barrel, the plunger having a push flange and the barrel defining an interior to movably receive the plunger, the barrel further including a base end including one or more lateral flanges and a tip end, the injector device comprising:
a first support defining a cavity surrounded by a peripheral edge into which the barrel of the syringe is perpendicularly insertable, wherein the base end of the barrel is supportable by the engagement between the peripheral edge of the first support and the one or more lateral flanges of the base end of the barrel;
a second support movably coupled to the first support, the second support having a guide surface that abuttably engages the push flange of the plunger when engaged therewith; and
at least two parallel guides movably coupling the second support to the first support.

8. An injector device for a syringe according to claim 1, wherein the opening has a U-shaped section defined by one of the sides of the first support.

9. An injector device for a syringe according to claim 1, wherein the cavity includes a circular opening with a diameter that is slightly greater than the diameter of the barrel of the syringe.

10. An injector device for a syringe according to claim 1, wherein the second support incorporates into its wall, opposite the first support, a recess into which the push flange is inserted to increase the positional stability of the plunger.

11. An injector device for a syringe according to claim 1, wherein at least one of the at least two parallel guides includes a stopper on an end thereof.

12. An injector device for syringes according to claim 1, wherein the at least two parallel guides include first and second opposite end portions, and wherein the first end portions are rigidly attached to the first support.

13. An injector device for syringes according to claim 12, further comprising a third support arranged in parallel with the first support, wherein the third support is rigidly coupled to the second end portions of the at least two parallel guides.

14. An injector device for syringes having a syringe plunger with a guide surface, the injector device enabling the syringe plunger to be moved gradually with little effort on the part of the patient or a third person, the syringe having a hollowed barrel to movably accept the syringe plunger, the hollowed barrel terminated by an end portion that includes a flange substantially longitudinally extending from the barrel, the injector device comprising:
a first support defining a cavity in which the barrel of the syringe is insertable perpendicularly to the first support, the flanges of the syringe resting on a peripheral edge of the cavity; and
a second central support on which the syringe plunger rests.

15. An injector device for syringes according to claim 14, further comprising:
parallel guides having first and second ends, the first end rigidly extending from the first support, wherein the second support is movably disposed along the parallel guides between said first support and said second ends of the parallel guides whereby the movement of the second support facilitates the push of the syringe plunger inside the hollowed barrel.

16. A syringe comprising:
a plunger having a push flange;
a barrel defining an interior to movably receive the plunger, the barrel further including a base end and a tip end, wherein the base end includes one or more lateral flanges;
an injector assist having a first support defining, at least in part, an opening surrounded by a peripheral edge into which the barrel of the syringe is insertable, wherein the base end of the barrel is supportable by the engagement between peripheral edge of the first support and the one or more lateral flanges of the base end of the barrel; and
a second support movably coupled to the first support, the second support having a guide surface that abuttably engages the push flange of the plunger when engaged therewith.

17. A syringe according to claim 16, further comprising:
at least two parallel guides movably coupling the second support to the first support.

18. A syringe according to claim 17, wherein the at least two parallel guides include first and second opposite end portions, and wherein the first end portions are rigidly attached to the first support.

19. A syringe according to claim 18, further comprising a third support arranged substantially parallel with the first support, wherein the third support is rigidly coupled to the second end portions of the at least two parallel guides.
